(19) United States
(12) Patent Application Publication
(10) Pub. No.: US 2021/0345885 A1
(43) Pub. Date: Nov. 11, 2021

(54) BIOLOGICAL INFORMATION MANAGEMENT APPARATUS, BIOLOGICAL INFORMATION MANAGEMENT METHOD, PROGRAM, AND RECORDING MEDIUM

(71) Applicant: NEC Solution Innovators, Ltd., Koto-ku, Tokyo (JP)

(72) Inventor: Kazuhide UMEDA, Tokyo (JP)

(73) Assignee: NEC Solution Innovators, Ltd., Koto-ku, Tokyo (JP)

(21) Appl. No.: 17/273,757

(22) PCT Filed: Sep. 6, 2019
(86) PCT No.: PCT/JP2019/035155

§ 371 Date: Mar. 5, 2021

(30) Foreign Application Priority Data
Sep. 6, 2018 (JP) 2018-167129

(51) Int. Cl. A61B 5/01; A61B 5/1171; A61B 5/0205; A61B 5/00

(52) U.S. Cl. CPC A61B 5/01; A61B 5/1176; A61B 5/02055; A61B 5/742; A61B 2503/20; A61B 5/746; A61B 5/7264; A61B 2560/02; A61B 5/7405

(57) ABSTRACT

An apparatus for biological information management including a storage and at least one processor. The processor is configured to: acquire a face image of a subject and a sensed biological information value of the subject, which are acquired at the same time, in association with each other, and perform face image authentication for the face image to identify the subject of the face image. The storage stores the biological information value associated with the face image in association with the subject. The processor is also configured to: determine a normal range of biological information values by cluster analysis from accumulated biological information values stored in the storage for each subject, determine whether or not a latest biological information value stored in the storage falls within the normal range, and output that the latest biological information value is abnormal when the latest biological information value falls outside the normal range.

### TECHNICAL FIELD

The present invention relates to a biological information management apparatus, a biological information management method, a program, and a recording medium.

### BACKGROUND ART

In food factories and the like, thorough hygiene management is necessary, and the entry of a worker into the factory is restricted, for example, in the case where he/she suffers a cold. However, the restriction on the entry only relies on, for example, self-reporting of the physical condition of the worker himself/herself.

By providing a thermometer such as that installed in the airport quarantine at the entrance of the factory, a worker having a higher body temperature than a certain level may be controlled to enter. However, since the body temperature differs among individuals, even if a certain threshold value is set, it is unknown, in fact, whether the body temperature is the body temperature affected by a bad physical condition or is the normal body temperature. For this reason, there is no other choice but to set the threshold value which is considered to be obviously abnormal temperature as in the case of influenza accompanied by high fever. The same applies to biological information other than the body temperature.

### SUMMARY OF INVENTION

With the foregoing in mind, it is an object of the present invention to provide a new system for managing biological information on an individual basis to detect an abnormality in biological information.

### Solution to Problem

In order to achieve the aforementioned object, the present invention provides a biological information management apparatus including: an information acquisition unit; an identification unit; a storage unit; a first determination unit; a second determination unit; and an output unit, wherein the information acquisition unit acquires a face image of a subject and a sensed biological information value of the subject, which are acquired at the same time, in association with each other, the identification unit performs face image authentication for the face image to identify the subject of the face image, the storage unit stores the biological information value associated with the face image in association with the subject, the first determination unit determines a normal range of biological information values by cluster analysis from accumulated biological information values stored in the storage unit for each subject, the second determination unit determines whether or not a latest biological information value stored in the storage unit falls within the normal range, and the output unit outputs that the latest biological information value is abnormal when the latest biological information value falls outside the normal range.

The present invention also provides a biological information management method, including the steps of: acquiring information; identifying; storing; first determining; second determining; and outputting, wherein the information-acquiring acquires a face image of a subject and a sensed biological information value of the subject, which are acquired at the same time, in association with each other, the identifying performs face image authentication for the face image to identify the subject of the face image, the storing stores the biological information value associated with the face image in association with the subject, the first determining determines a normal range of biological information values by cluster analysis from accumulated biological information values stored in the storing for each subject, the second determining determines whether or not a latest biological information value stored in the storing falls within the normal range, and the outputting outputs that the latest biological information value is abnormal when the latest biological information value falls outside the normal range.

The present invention also provides a program for a computer to execute the biological information management method according to the present invention.

The present invention also provides a computer readable recording medium with the program according to the present invention.

### Advantageous Effects of Invention

According to the present invention, biological information is managed on an individual basis to detect an abnormality in biological information. Therefore, according to the present invention, an abnormality in a body temperature in consideration of the individual difference can be detected in a simpler manner.

### BRIEF DESCRIPTION OF DRAWINGS

FIG. 1 is a block diagram showing an example of a body temperature management apparatus according to the first example embodiment.

FIG. 2 is a block diagram showing an example of the body temperature management apparatus according to the first example embodiment.

FIG. 3 is a flowchart showing an example of a body temperature management method according to the first example embodiment.

### DESCRIPTION OF EXAMPLE EMBODIMENTS

Embodiments of the present invention will be described. Note here that the present invention is not limited to the following example embodiments. In the drawings, identical parts are indicated with identical reference signs. Each example embodiment can be described with reference to the descriptions of other example embodiments, unless otherwise specified, and the configurations of the example embodiments may be combined, unless otherwise specified.

In the present invention, the biological information is information relating to an organism, and is not particularly limited. Examples of the biological information value include a body temperature, a pulse, a heart rate, a blood glucose level, and a blood pressure. The organism is not particularly limited, and examples thereof include humans and non-human animals.

### First Example Embodiment

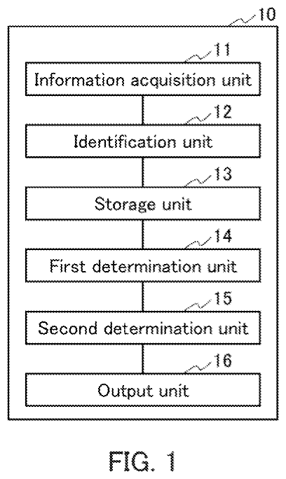

In the present example embodiment, a case in which the biological information value is a body temperature will be described as an example. The present invention, however, is not limited thereto. FIG. 1 is a block diagram showing a configuration of an example of a body temperature management apparatus 10 according to the present example embodiment. The body temperature management apparatus 10 includes an information acquisition unit 11, an identification unit 12, a storage unit 13, a first determination unit 14, a second determination unit 15, and an output unit 16. The body temperature management apparatus 10 is also referred to as a body temperature management system, for example.

The body temperature management apparatus 10 may be, for example, a single body temperature management apparatus including the above-described components, or may be a body temperature management apparatus to which the components are connectable via a communication network.

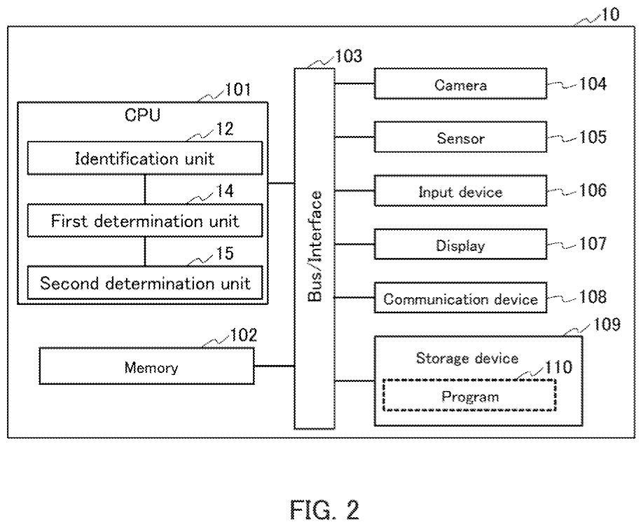

FIG. 2 illustrates a block diagram of the hardware configuration of the body temperature management apparatus 10. The body temperature management apparatus 10 includes, for example, a central processing unit (CPU) 101, a memory 102, an interface (I/F) 103, a camera 104, a sensor 105, an input device 106, a display 107, a communication device 108, and a storage device 109. The components of the body temperature management apparatus 10 are connected to each other via a I/F103, for example. In the hardware configuration, communication between internal circuits is connected by a bus.

The CPU 101 serves to control the entire body temperature management apparatus 10. In the body temperature management apparatus 10, the CPU 101 executes a program of the present invention and other programs, and reads and writes various pieces of information, for example. Specifically, for example, the CPU 101 of the body temperature management apparatus 10 functions as the identification unit 12, the first determination unit 14, and the second determination unit 15.

The I/F 103 connects, for example, between the respective functional components such as the CPU 101, the memory 102, and the like. The I/F 103 can also be connected, for example, to an external device. The external device may be, for example, a terminal or the like. The body temperature management apparatus 10 can be connected to a communication network by a communication device 108 connected to the I/F103, and can also be connected to the external device via the communication network.

The memory 102 includes, for example, a main memory, and the main memory is also referred to as a main storage device. When the CPU101 performs processing, the memory 102 reads various kinds of operation programs 110 such as the program of the present invention stored in an auxiliary storage device to be described below, and the CPU101 receives data from the memory 102 and executes the program 110. The main memory is, for example, a random access memory (RAM). The memory 102 further includes, for example, a read-only memory (ROM).

The storage device 109 is also referred to as, for example, a so-called auxiliary storage device relative to the main memory (main storage device). The storage device 109 includes, for example, a storage medium and a drive for reading from and writing to the storage medium. The storage medium is not particularly limited, and may be, for example, a built-in type or an external type, and examples thereof include a hard disk (HD), a floppy® disk (FD), a CD-ROM, a CD-R, a CD-RW, an MO, a DVD, a flash memory, and a memory card. The drive is not particularly limited. The storage device 109 may be, for example, a hard disk drive (HDD) in which the storage medium and the drive are integrated. For example, the storage device 109 may store the operation program 110 as described above. The storage device 109 may store the operation program 110 in association with data such as the body temperature of the subject to be described below, for example.

The body temperature management apparatus 10 further includes, for example, a camera 104, a sensor 105, an input device 106, and a display 107. The input device 106 is, for example, a touch panel or the like. The display 107 serves as, for example, the output unit 16. Examples of the display 107 include an LED display and a liquid crystal display.

The information acquisition unit 11 acquires a face image of a subject and a sensed body temperature of the subject, which are acquired at the same time, in association with each other. The information acquisition unit 11 itself may be, for example, an imaging unit that captures a face image of the subject and a body temperature acquisition unit that acquires the body temperature of the subject, or may be an input unit that inputs image information of the captured face image and information of the acquired body temperature. The imaging unit is, for example, a camera 104. The body temperature acquisition unit is, for example, a sensor 105. For example, the sensor 105 may sense the body temperature by contact with the subject, or may sense the body temperature without contact, and specific examples of the sensor include a thermometer and a thermo camera. The thermometer can sense the body temperature of the subject, for example, by placing the subject's hand on the thermometer. The thermo camera acquires an image of the subject by infrared rays, for example, and can sense the body temperature of the subject from the image. In the information acquisition unit 11, the imaging unit and the body temperature acquisition unit may be integrated, for example. Thus, for example, acquisition of the face image and acquisition of the body temperature can be performed simultaneously. Specifically, the information acquisition unit 11 is a camera capable of sensing visible light and infrared rays, and can acquire a face image of the subject by the visible light and acquire the body temperature of the subject by the infrared rays, for example.

Note that, as described above, the biological information value is not limited to the body temperature, and when the biological information value is, for example, a pulse, a heart rate, a blood glucose value, a blood pressure, or the like, the information acquisition unit 11 is not particularly limited, and for example, known devices for acquiring these biological information values can be used.

When the information acquisition unit 11 is the input unit, the body temperature management apparatus 10 may be electrically connected to, for example, a camera and a sensor outside the apparatus, and image information captured by the camera and body temperature information acquired by the sensor may be input. The connection between the body temperature management apparatus 10, the camera, and the sensor is not particularly limited, and may be, for example, wired or wireless. In this case, the body temperature management apparatus 10, the camera, and the sensor can be connected to each other via, for example, a communication network. The communication network is not particularly limited, and may be a well-known communication network, which may be wired or wireless, and specific examples thereof include an Internet line, a telephone line, a Local Area Network (LAN), and a Wireless Fidelity (WiFi).

The installation locations of the camera 104 and the sensor 105 in the information acquisition unit 11 are not particularly limited as long as they can acquire the face image and the body temperature of the subject, respectively, and they can be installed, for example, at the entrance of a predetermined area. The camera and sensor outside the apparatus can also be installed in the same manner. Examples of the area include a factory such as a food factory and an airport. The installation locations of the camera 104 and the sensor 105 in the area are not particularly limited. The camera 104 and the sensor 105 may be installed at an entrance of the area, for example, at locations where a face image and a body temperature of a subject who intends to enter from the entrance can be acquired. The installation location of the sensor 105 can be appropriately set in accordance with, for example, the type of the sensor 105, a body part at which a body temperature is acquired, and the like. The number of cameras 104 and sensors 105 is not particularly limited, and may be, for example, one or two or more. When two or more sensors 105 are installed, the types of the sensor 105 and the body parts at which a body temperature is acquired are preferably the same, for example. However, the present invention is not limited thereto, and two or more sensors 105 may be different types of sensors or may acquire body temperatures at different body parts. When the body temperature management apparatus 10 includes two or more sensors 105, the body temperature management apparatus 10 may further include, for example, a body temperature correction unit, and the body temperature correction unit may correct the measurement error of the body temperature due to, for example, the type and installation location of the sensors 105, the body part at which a body temperature is acquired, and the like.

For example, the information acquisition unit 11 may acquire, in addition to the face image of the subject and the body temperature of the subject, the shooting date and time in association therewith. Thereby, the body temperature management apparatus 10 can manage the body temperature of the subject based on, for example, the shooting date and time.

It is known that the body temperature fluctuates depending on the time, the air temperature, and the like even in the same person, for example. For this reason, for example, the measured value of the body temperature may be corrected by the body temperature correction unit or the like based on the shooting date and time, the season, the air temperature, and the like.

The identification unit 12 performs face image authentication for the face image to identify the subject of the face image. The face image authentication is not particularly limited, and conventionally known face authentication methods such as a method based on similarity calculation between the face images, a method using a ratio of distances between feature points such as eyes, nose, and mouth, a feature amount such as an orientation of the line connecting the feature points, and the like can be used. For the face image authentication, for example, a face authentication system such as Neo Face KAOATO (product name) can be used.

The subject of the face image authentication may be, for example, a person registered in advance or an unspecified person. When the subject is a person registered in advance, for example, the face image for authentication of the subject may be stored in advance in the storage device 108 of the body temperature management apparatus 10, and the identification unit 12 may perform the face image authentication with respect to the face image for authentication and the face image acquired by the information acquisition unit 11.

The storage unit 13 stores the body temperature associated with the face image in association with the subject. For example, the storage unit 13 may further store data such as the acquisition date and time of the body temperature, the normal body temperature range of the subject to be described below, the face image for authentication of the subject, and the like in association with the subject.

The first determination unit 14 determines the normal body temperature range by cluster analysis from the accumulated body temperatures stored in the storage unit for each subject. The cluster analysis is a method of classifying data by, for example, collecting data similar to each other from a set of data to form clusters. The cluster analysis can be performed, for example, by a known method. Parameters and the like in the cluster analysis can be set as appropriate. By the cluster analysis, for example, even when an abnormal body temperature value is included in the accumulated body temperatures, the normal body temperature range can be determined without being affected by the abnormal body temperature value.

For example, the first determination unit 14 classifies the accumulated body temperature data into clusters, and determines the normal body temperature range from the clusters. For example, the first determination unit 14 may determine a cluster having a larger number of data included in the cluster as a cluster including the normal body temperature range, or may determine a cluster including body temperature data closer to a general human body temperature as a cluster including the normal body temperature range. Further, for example, at the time of registration of the subject, after checking that the physical condition of the subject is normal, the body temperature may be measured and stored as the normal time body temperature, and the cluster including the normal time body temperature may be determined as the cluster including the normal body temperature range. In addition, it is preferable to measure the actual surface temperature (e.g., average value) at a site of a factory or the like in a state where the body temperature of the subject is normal (normal temperature), and to store the actual surface temperature together with the normal time body temperature at the time of registration of the subject. The actual surface temperature is, for example, a measurable skin surface temperature at a location where a body temperature of a subject is actually measured by the sensor 105 such as the thermo camera. Thus, if the average value of the normal time actual surface temperature (normal temperature time actual surface temperature) is stored, for example, when the subject has a high fever, the actual surface temperature of the subject deviates from the average value of the normal time actual surface temperature, so that when the actual surface temperature deviated in this manner is detected, an alert may be output as an abnormal value. As the actual surface temperature, for example, the upper limit value and the lower limit value can be set for each site.

The normal temperature range is not particularly limited, and may be, for example, a range of body temperatures included in the cluster, or may be determined from the cluster by a known statistical method. In the latter case, specifically, for example, the average value, the median, and the mode value or the like of the body temperatures included in the cluster is calculated as a reference body temperature, and the normal body temperature range can be set in a predetermined range based on the reference body temperature. The predetermined range based on the reference body temperature is not particularly limited and can be set appropriately.

The second determination unit 15 determines whether or not the latest body temperature stored in the storage unit 13 falls within the normal body temperature range.

When the latest body temperature falls outside the normal body temperature range, the output unit 16 outputs that the latest body temperature is abnormal. The output may be, for example, the generation of an alarm sound or the display of a warning. The output may be made to an external terminal via, for example, a communication network, or may be output to a display when the body temperature management apparatus 10 includes the display 107. Thus, for example, when the subject shows an abnormality in body temperature such as high fever, the abnormality can be detected.

The output unit 16 may be, for example, an entry control unit for controlling the entry of the subject into the predetermined area. That is, for example, if the latest body temperature stored in the storage unit 13 falls within the normal body temperature range, the output unit 16 may unlock the entrance to the area for the subject, and if the latest body temperature falls outside the normal body temperature range, the output unit 16 may keep locking the entrance to the area against the subject. The output unit 16 may generate an alarm sound, display a warning, and the like when the entry of the subject into the area is detected in the state where the latest body temperature stored in the storage unit 13 falls outside the normal body temperature range. Thereby, for example, when the subject shows an abnormality in body temperature such as high fever, entry of the subject into the area can be restricted.

Next, the body temperature management method of the present example embodiment will be described. The body temperature management method of the present example embodiment can be performed using the body temperature management apparatus 10 shown in FIGS. 1 and 2, for example. Note that the body temperature management method of the present example embodiment is not limited to the use of the body temperature management apparatus 10 shown in these drawings. Regarding the body temperature management apparatus 10 described above, reference can be made to the description of the body temperature management method of the present example embodiment.

The information-acquiring acquires a face image of a subject and a sensed body temperature of the subject, which are acquired at the same time, in association with each other. The information-acquiring can be performed by, for example, the information acquisition unit 11 of the body temperature management apparatus 10.

The identifying performs face image authentication for the face image to identify the subject of the face image. The identifying can be performed, for example, by the identification unit 12 of the body temperature management apparatus 10.

The storing stores the body temperature associated with the face image in association with the subject. The storing can be performed by, for example, the storage unit 13 of the body temperature management apparatus 10.

The first determining determines a normal body temperature range from body temperatures stored in the storage unit for each subject. The first determining may be performed every time the body temperature is newly stored in the storing or may be performed every predetermined period of time, for example. The first determining can be performed by, for example, the first determination unit 14 of the body temperature management apparatus 10.

The outputting outputs that the body temperature is abnormal when the latest body temperature stored in the storage unit falls outside the normal body temperature range. The outputting can be performed by, for example, the output unit 16 of the body temperature management apparatus 10.

The outputting may be, for example, a step of controlling entry of the subject into the predetermined area. In the entry-controlling, for example, if the latest body temperature stored in the storing falls within the normal body temperature range, the entrance to the area may be unlocked for the subject, and if the latest body temperature falls outside the normal body temperature range, the entrance to the area may be locked for the subject. In the entry-controlling, for example, when the entry of the subject into the area is detected in the state where the latest body temperature stored in the storing falls outside the normal body temperature range, an alarm sound may be generated, a warning may be displayed, and the like.

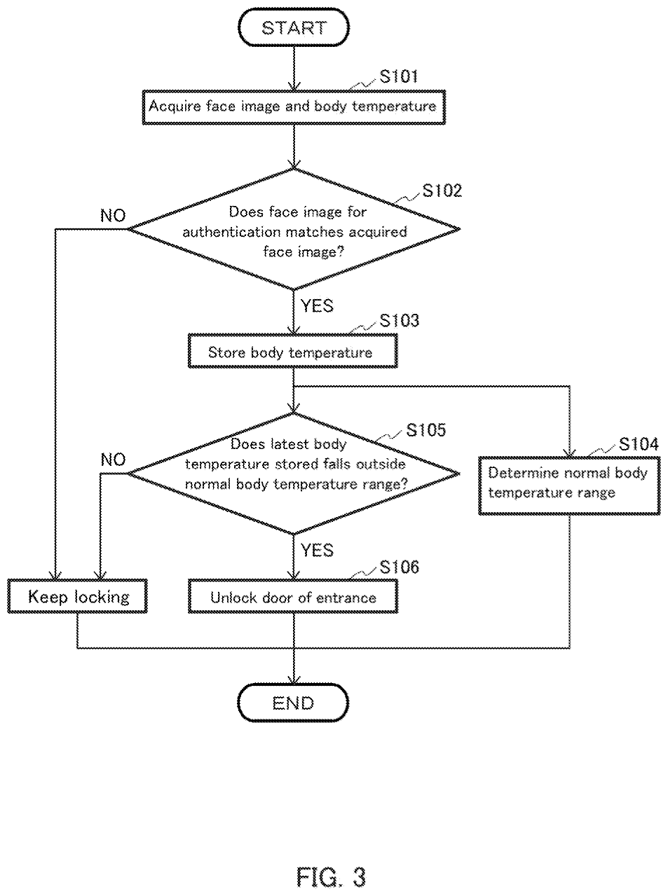

Next, the body temperature management method of the present example embodiment will be described in more detail with reference to the flowchart of FIG. 3. Note that the body temperature management method of the present invention is not limited to these examples.

In the description using the flowchart, a case in which the body temperature management apparatus 10 is used in a food factory will be described as an example. A door is provided at the entrance of the food factory, and the door is, for example, an automatic door. A camera for capturing a face image and a sensor for measuring a body temperature are provided outside the entrance.

Face images of employees of the food factory are stored in advance as face images for authentication in the storage unit 13 of the body temperature management apparatus 10.

When the employee arrives in front of the entrance door of the food factory, the camera captures an image of the employee's face and the sensor measures the employee's body temperature. Then, the information acquisition unit 11 of the body temperature management apparatus 10 acquires the captured face image of the employee and the body temperature of the employee in association with each other (S101).

Next, the identification unit 12 of the body temperature management apparatus 10 determines whether or not the face image for authentication stored in the storage unit 13 matches the acquired face image of the employee, and identifies the employee when the acquired face image of the employee matches one of the plurality of face images for authentication (S102).

When the employee is identified in the step (S102), the storage unit 13 of the body temperature management apparatus 10 stores the body temperature associated with the acquired face image in association with the identified employee (S103). On the other hand, if the employee is not identified in the step (S102), the step (S103) ends without storing the body temperature. The present invention, however, is not limited to this, and if the employee is not identified in the step (S102), the acquired face image may be newly stored in the step (S103), and the body temperature associated with the face image may be stored in association with the acquired face image.

In addition, the first determination unit 14 of the body temperature management apparatus 10 determines the normal body temperature range for each employee by cluster analysis from accumulated body temperatures stored in the storage unit 13 (S104). The order of performing the step (S104) is not particularly limited, and for example, after the step (S103), before the step (S105), after the step (S105), before the step (S106), or after the step (S106).

Then, the second determination unit 15 of the body temperature management apparatus 10 determines whether or not the latest body temperature stored in the storage unit falls within the normal body temperature range (S105). If the latest body temperature falls within the normal body temperature range, the output unit 16 unlocks the door of the entrance (S106) and ends the procedure (END). On the other hand, if the latest body temperature falls outside the normal body temperature range, the door of the entrance is kept locked, and the procedure is ended without unlocking (END).

As described above, the body temperature management apparatus 10 can manage a body temperature on an individual basis and detect an abnormality of the body temperature, for example. Therefore, by managing the body temperature of the employees of the food factory by using the body temperature management apparatus 10, even when it is not the case of showing an obvious abnormal body temperature as in the case of the onset of influenza accompanied by high fever, it is possible to detect the abnormality in the body temperature of the employees due to the initial cold or the like, and thereby it is possible to further prevent the spread of infection in the factory. Thus, the body temperature management apparatus 10 can be suitably used in the food factory from the viewpoint of hygiene management. The present invention, however, is not limited thereto, and since the body temperature management apparatus 10 can prevent a decrease in attention of the employees and an occurrence of an accident due to having fever, it can be suitably used even in various factories other than the food factory described above, for example.

In addition, the body temperature management apparatus 10 can be used, for example, for the body temperature management of travelers in airports. In this case, for example, cameras for capturing face images of the travelers and sensors for measuring body temperatures are provided at immigration sites of airports in each country.

The body temperature management apparatus 10 acquires a face image and a body temperature, which are acquired at an airport of each country, in association with each other. Then, the body temperature management apparatus 10 stores the body temperature associated with the face image for each traveler and determines the normal body temperature range of the traveler from the stored body temperatures.

When the latest body temperature of the traveler stored in the storage unit falls outside the normal body temperature range, the body temperature management apparatus 10 generates an alarm sound and outputs that the traveler's body temperature is abnormal.

As described above, according to the body temperature management apparatus 10, for example, even when it is not the case of showing an obvious abnormal body temperature at an airport, it is possible to detect the abnormality in the body temperature of the traveler. Therefore, for example, even before the body temperature becomes very high, it is possible to detect a traveler who is highly likely to be infected, and by preventing the traveler from entering the country, it is possible to further prevent the spread of infection in each country.

While the biological information management apparatus and the biological information management method of the present invention have been described with reference to a case in which the biological information value is a body temperature as an example, the biological information value is not limited to the body temperature. The form and the installation location of the information acquisition unit 11, and the parameters and the like in the cluster analysis by the first determination unit 14 can be appropriately selected according to the type and the like of the biological information.

### Second Example Embodiment

The program of the present example embodiment is a program for a computer to execute the body temperature management method of each of the example embodiments. The program of the present example embodiment may be recorded on, for example, a computer readable recording medium. The recording medium is not particularly limited, and examples thereof include a read-only memory (ROM), a hard disk (HD), an optical disk, and a floppy® disk (FD).

While the present invention has been described above with reference to illustrative example embodiments, the present invention is by no means limited thereto. Various changes and variations that may become apparent to those skilled in the art may be made in the configuration and specifics of the present invention without departing from the scope of the present invention.

This application claims priority from Japanese Patent Application No. 2018-167129 filed on Sep. 6, 2018. The entire subject matter of the Japanese Patent Application is incorporated herein by reference.

### Supplementary Notes

Some or all of the above example embodiments may be described as in the following Supplementary Notes, but are not limited thereto.

(Supplementary Note 1)

A biological information management apparatus including:
an information acquisition unit;
an identification unit;
a storage unit;
a first determination unit;
a second determination unit; and
an output unit, wherein
the information acquisition unit acquires a face image of a subject and a sensed biological information value of the subject, which are acquired at the same time, in association with each other,
the identification unit performs face image authentication for the face image to identify the subject of the face image,
the storage unit stores the biological information value associated with the face image in association with the subject,
the first determination unit determines a normal range of biological information values by cluster analysis from accumulated biological information values stored in the storage unit for each subject,
the second determination unit determines whether or not a latest biological information value stored in the storage unit falls within the normal range, and
the output unit outputs that the latest biological information value is abnormal when the latest biological information value falls outside the normal range.

(Supplementary Note 2)

The biological information management apparatus according to Supplementary Note 1, wherein
the information acquisition unit acquires at least one selected from the group consisting of a body temperature, a pulse, a heart rate, a blood glucose level, and a blood pressure as the biological information value.

(Supplementary Note 3)

The biological information management apparatus according to Supplementary Note 1 or 2, wherein
the information acquisition unit acquires a body temperature as the biological information value.

(Supplementary Note 4)

The biological information management apparatus according to Supplementary Note 3, wherein
the information acquisition unit includes a thermo camera, and
the thermo camera senses a body temperature of the subject.

(Supplementary Note 5)

The biological information management apparatus according to any one of Supplementary Notes 1 to 4, wherein
the information acquisition unit acquires a face image of a subject, a sensed biological information value of the subject, and an acquisition date and time, which are acquired at the same time, in association with each other, and
the storage unit stores the biological information value and the acquisition date and time associated with the face image in association with the subject.

(Supplementary Note 6)

The biological information management apparatus according to any one of Supplementary Notes 1 to 5, wherein
output by the output unit is at least one selected from the group consisting of generation of an alarm sound, display of a warning, and control of entry of the subject into a predetermined area.

(Supplementary Note 7)

The biological information management apparatus according to any one of Supplementary Notes 1 to 6, wherein
the area is at least one of a factory or an airport.

(Supplementary Note 8)

A biological information management method, including the steps of:
acquiring information;
identifying;
storing;
first determining;
second determining; and
outputting, wherein
the information-acquiring acquires a face image of a subject and a sensed biological information value of the subject, which are acquired at the same time, in association with each other,
the identifying performs face image authentication for the face image to identify the subject of the face image,
the storing stores the biological information value associated with the face image in association with the subject,
the first determining determines a normal range of biological information values by cluster analysis from accumulated biological information values stored in the storing for each subject,
the second determining determines whether or not a latest biological information value stored in the storing falls within the normal range, and
the outputting outputs that the latest biological information value is abnormal when the latest biological information value falls outside the normal range.

(Supplementary Note 9)

The biological information management method according to Supplementary Note 8, wherein
in the information-acquiring, at least one selected from the group consisting of a body temperature, a pulse, a heart rate, a blood glucose level, and a blood pressure is selected as the biological information value.

(Supplementary Note 10)

The biological information management method according to Supplementary Note 8 or 9, wherein
in the information-acquiring, a body temperature is acquired as the biological information value.

(Supplementary Note 11)

The biological information management method according to Supplementary Note 10, wherein
in the information-acquiring, a body temperature of the subject is sensed by a thermo camera.

(Supplementary Note 12)

The biological information management method according to any one of Supplementary Notes 8 to 11, wherein
in the information-acquiring, a face image of a subject, a sensed biological information value of the subject, and an acquisition date and time, which are acquired at the same time, are acquired in association with each other, and
   in the storing, the biological information value and the acquisition date and time associated with the face image are stored in association with the subject.

(Supplementary Note 13)

The biological information management method according to any one of Supplementary Notes 8 to 12, wherein,
in the outputting, output is at least one selected from the group consisting of generation of an alarm sound, display of a warning, and control of entry of the subject into a predetermined area.

(Supplementary Note 14)

The biological information management method according to any one of Supplementary Notes 8 to 13, wherein
the area is at least one of a factory or an airport.

(Supplementary Note 15)

A program for a computer to execute the biological information management method according to any one of Supplementary Notes 8 to 14.

(Supplementary Note 16)

A computer readable recording medium with the program according to Supplementary Note 15.

### INDUSTRIAL APPLICABILITY

According to the present invention, a body temperature is managed on an individual basis to detect an abnormality in a biological information value such as the body temperature. Therefore, according to the present invention, an abnormality in a biological information value in consideration of the individual difference can be detected in a simpler manner.
10: body temperature management apparatus
11: information acquisition unit
12: identification unit
13: storage unit
14: first determination unit
15: second determination unit
16: output unit

What is claimed is:

1-16. (canceled)

17. An apparatus for biological information management comprising a storage and at least one processor:
the processor is configured to acquire a face image of a subject and a sensed biological information value of the subject, which are acquired at the same time, in association with each other,
the processor is configured to perform face image authentication for the face image to identify the subject of the face image,
the storage stores the biological information value associated with the face image in association with the subject,
the processor is configured to determine a normal range of biological information values by cluster analysis from accumulated biological information values stored in the storage for each subject,
the processor is configured to determine whether or not a latest biological information value stored in the storage falls within the normal range, and
the processor is configured to output that the latest biological information value is abnormal when the latest biological information value falls outside the normal range.

18. The apparatus according to claim 17, wherein
the processor is configured to acquire at least one selected from the group consisting of a body temperature, a pulse, a heart rate, a blood glucose level, and a blood pressure as the biological information value.

19. The apparatus according to claim 17, wherein
the processor is configured to acquire a body temperature as the biological information value.

20. The apparatus according to claim 19, wherein
the apparatus includes a thermo camera, and
the thermo camera senses a body temperature of the subject.

21. The biological information management apparatus according to claim 17, wherein
the processor is configured to acquire a face image of a subject, a sensed biological information value of the subject, and an acquisition date and time, which are acquired at the same time, in association with each other, and
the storage stores the biological information value and the acquisition date and time associated with the face image in association with the subject.

22. The apparatus according to claim 17, wherein
the processor is configured to output at least one selected from the group consisting of generation of an alarm sound, display of a warning, and control of entry of the subject into a predetermined area.

23. The apparatus according to claim 17, wherein
the area is at least one of a factory or an airport.

24. A computer-implemented method for a biological information management, comprising:
acquiring a face image of a subject and a sensed biological information value of the subject, which are acquired at the same time, in association with each other,
the identifying performs face image authentication for the face image to identify the subject of the face image,
storing the biological information value associated with the face image in association with the subject,
determining a normal range of biological information values by cluster analysis from accumulated biological information values stored in the storing for each subject,
determining whether or not a latest biological information value stored in the storing falls within the normal range, and
outputting that the latest biological information value is abnormal when the latest biological information value falls outside the normal range.

25. The method according to claim 24, wherein
in the information-acquiring, at least one selected from the group consisting of a body temperature, a pulse, a heart rate, a blood glucose level, and a blood pressure is selected as the biological information value.

26. The method according to claim 24, wherein
acquiring a body temperature as the biological information value.

27. The method according to claim 26, wherein
sensing a body temperature of the subject by a thermo camera.

28. The biological information management method according to claim 24, wherein
acquiring a face image of a subject, a sensed biological information value of the subject, and an acquisition date and time, which are acquired at the same time, in association with each other, and
storing the biological information value and the acquisition date and time associated with the face image in association with the subject.

29. The method according to claim 24, wherein,
outputting at least one selected from the group consisting of generation of an alarm sound, display of a warning, and control of entry of the subject into a predetermined area.

30. The method according to claim 24, wherein
the area is at least one of a factory or an airport.

31. A computer readable recording medium with a program for a computer to execute the biological information management method according to claim 24.
